(19) United States
(12) Patent Application Publication
(10) Pub. No.: US 2026/0060905 A1
(43) Pub. Date: Mar. 5, 2026

(54) USE OF POLYMER DOTS FOR ENHANCING HAIR GROWTH OR PREVENTING HAIR LOSS

(71) Applicant: China Medical University, Taichung City (TW)

(72) Inventors: Tzong-Yuan Juang, Taichung City (TW); Chang-Cheng Chang, Taichung City (TW); Siao-Cian Fan, Kaohsiung City (TW); Ching-Hsuan Lin, Taichung City (TW)

(73) Assignee: China Medical University, Taichung City (TW)

(21) Appl. No.: 19/313,831

(22) Filed: Aug. 28, 2025

(30) Foreign Application Priority Data
Aug. 29, 2024 (TW) 113132553

(51) Int. Cl. A61K 8/19; A61K 8/84; A61Q 7/00

(52) U.S. Cl. CPC A61K 8/19; A61K 8/84; A61Q 7/00; A61K 2800/413

(57) ABSTRACT

There is a great amount of people suffering from hair loss or baldness due to stress, hormonal changes, male baldness, side effects of drugs or vaccines, aging or immune system problems; and there are also some people who do not have the aforementioned problems but still have a need for increasement of hair volume. To solve the aforementioned problems, a use of polymer dot in manufacture of a composition for the enhancement of hair growth or the prevention of hair loss is provided, in order to enhance hair growth of a patient with hair loss or baldness and to prevent hair loss of a non-patient. The polymer dot includes a carbon dot.

### BACKGROUND OF THE INVENTION

### 1. Field of the Invention

The present disclosure relates to the use of polymer dots, and more particularly to the use of polymer dots for promoting hair growth or preventing hair loss.

### 2. Description of the Prior Art

Hair is one of the major features of mammals and plays an important role in several physiological functions. The hair follicle undergoes a metabolic cyclical process mainly including anagen (the formation of hair follicles and hair shafts), catagen (apoptosis-driven degeneration), and telogen (hair loss and dormancy) phases. Hair follicle and hair regeneration is governed by complex epithelial-mesenchymal interactions (EMIs).

An increasing number of individuals are experiencing hair loss or baldness. The underlying causes are multifactorial and include stress, hormonal fluctuations, androgenetic alopecia (also known as hereditary hair loss), side effects of medications or vaccines, aging, and immune-related disorders (e.g., excessive immune responses leading to sustained inflammation and follicular damage). Hair loss or baldness involves complex and dynamic molecular mechanisms. Moreover, some individuals may have a need to increase hair volume.

However, effective non-invasive strategies for addressing the problems of hair loss or baldness in hair loss or baldness patients or non-patients remain lacking.

Accordingly, there is a need in the art for providing approaches that may effectively enhance hair growth and prevent hair loss capable of overcoming the above-mentioned problems.

### SUMMARY OF THE INVENTION

To address the aforementioned problems, the present disclosure provides a use of polymer dots in manufacture of a composition for enhancing hair growth or preventing hair loss. The polymer dot may include a carbon dot.

In summary, the polymer dot of the present disclosure may not only solve the problems in the art associated with enhancing hair growth and preventing hair loss, but also provide the following functions and effects in some embodiments,:
(1) The polymer dot of the present disclosure has advantages of highly dispersible in water, possessing abundant surface functional groups, exhibiting excellent biocompatibility, and easy to use.
(2) The polymer dot of the present disclosure may effectively scavenge free radicals and peroxides.
(3) The polymer dot of the present disclosure meets the requirement of nanozyme characteristics.
(4) The polymer dot of the present disclosure demonstrates superior efficacy in enhancing hair growth compared to those of the commercially available products.
(5) The polymer dot of the present disclosure may induce epithelial-mesenchymal interactions (EMI) and accelerate the transition of hair follicles from the telogen phase to the anagen phase, thereby inducing hair follicle regeneration.
(6) The polymer dot of the present disclosure may effectively increase the number of hair follicles.
(7) The polymer dots disclosed herein may optionally be used in combination with other therapeutic drugs or biological agents as needed

The objectives of the present disclosure will no doubt become understandable to those of ordinary skill in the art after reading the following detailed description of the embodiments that are illustrated in the various figures and drawings.

### BRIEF DESCRIPTION OF THE DRAWINGS

FIG. 1A is a schematic diagram illustrating a preparation process of a hyperbranched polymer dot according to an embodiment of the present disclosure.

FIG. 1B is a schematic diagram illustrating a preparation process of a hyperbranched polymer dot according to an embodiment of the present disclosure.

The upper panels of FIG. 2 (top) are graph showing particle size distribution of hyperbranched polymer dots according to an embodiment of the present disclosure.

The lower panels of FIG. 2 (bottom) are images showing the morphology of hyperbranched polymer dots according to an embodiment of the present disclosure.

The left panel of FIG. 3 is a bar graph illustrating evaluation of cytotoxicity of hyperbranched polymer dots according to an embodiment of the present disclosure.

The right panels of FIG. 3 are a microscopy image showing cellular uptake of hyperbranched polymer dots according to an embodiment of the present disclosure.

FIG. 4 is a schematic diagram illustrating a preparation process of a hyperbranched polymer dot according to an embodiment of the present disclosure.

FIG. 5 are images showing the dorsal surface of control or treatment group after 18 days of treatment with phosphate-buffered saline or hyperbranched polymer dots, respectively, according to one embodiment of the present disclosure.

FIG. 6 is a line graph showing the body weight changes in control or treatment group after treatment with phosphate-buffered saline or hyperbranched polymer dots, respectively, according to one embodiment of the present disclosure.

FIG. 7A are images showing the dorsal surface of control or treatment group after 15 days of treatment with phosphate-buffered saline or hyperbranched polymer dots, respectively, according to one embodiment of the present disclosure.

FIG. 7B is a bar graph illustrating hair coverage rates in control or treatment group after 15 days of treatment with phosphate-buffered saline or hyperbranched polymer dots, respectively, according to one embodiment of the present disclosure.

FIG. 7C is a bar graph showing skin color scores of the dorsal region in control or treatment group after 15 days of treatment with phosphate-buffered saline or hyperbranched polymer dots, respectively, according to one embodiment of the present disclosure.

FIG. 7D is a line graph showing skin color scores of the dorsal region of control or treatment group after 15 days of treatment with phosphate-buffered saline or hyperbranched polymer dots, respectively, according to one embodiment of the present disclosure.

FIG. 8 are images showing the dorsal surface of control or treatment group after treatment with phosphate-buffered saline or hyperbranched polymer dots, respectively, according to one embodiment of the present disclosure.

FIG. 9 are images showing the dorsal surface of control or treatment group after treatment with phosphate-buffered saline or hyperbranched polymer dots, respectively, according to one embodiment of the present disclosure.

FIG. 10 are optical coherence tomography (OCT) images of dorsal region of skin of control or treatment group after treatment with phosphate-buffered saline or hyperbranched polymer dots, respectively, according to one embodiment of the present disclosure.

FIG. 11 are histological images of dorsal region of skin of control or treatment group after treatment with phosphate-buffered saline or hyperbranched polymer dots, respectively, according to one embodiment of the present disclosure.

### DETAILED DESCRIPTION

The following descriptions of the embodiments illustrate implementations of the present disclosure, and those skilled in the art of the present disclosure can readily understand the advantages and effects of the present disclosure in accordance with the contents herein. However, the embodiments of the present disclosure are not intended to limit the scope of the present disclosure. The present disclosure can be practiced or applied by other alternative embodiments, and every detail included in the present disclosure can be changed or modified in accordance with different aspects and applications without departing from the essentiality of the present disclosure.

The features such as a ratio, structure, and dimension shown in drawings accompanied with the present disclosure are simply used to cooperate with the contents disclosed herein for those skilled in the art to read and understand the present disclosure, rather than to limit the scope of implementation of the present disclosure. Thus, in the case that does not affect the purpose of the present disclosure and the effect brought by the present disclosure, any change in proportional relationships, structural modification, or dimensional adjustment should fall within the scope of the technical contents disclosed herein.

As used herein, “comprising”, “including”, or “having” a specific element, unless otherwise specified, may include other elements such as materials, components, structures, steps, or connection relationships rather than exclude those elements.

Unless otherwise specified, the singular forms “a/an” and “the” used herein also include plural forms, and the terms “or”and “and/or”used herein are interchangeable.

The numeral ranges used herein are inclusive and combinable, any numeral value that falls within the numeral scope herein could be taken as a maximum or minimum value to derive the sub-ranges therefrom. For example, the numeral range “3 nm to 5 nm” includes any sub-ranges between the minimum value of 3 nm to the maximum value of 5 nm, such as the sub-ranges from 3 nm to 4.5 nm, 3.5 nm to 4 nm, and 4.2 nm to 4.8 nm and so on. In addition, a plurality of numeral values used herein can be optionally selected as maximum and minimum values to derive numerical ranges. For instance, the numerical ranges of 3 nm to 4.2 nm, 4.2 to 5 nm, or 3 nm to 5 nm can be derived from the numeral values of 3 nm, 4.2 nm, and 5 nm.

Unless otherwise specified, the term “nanoparticle” as used herein is interchangeable with “polymer dot,” “hyperbranched polymer dots,” “hyperbranched polymer,” “polymer dots,” “carbon dot,” “polymer nanocarbon dot,” “nanodot,” “nanocluster,” or “carbon nanodot. ” Unless otherwise specified, the term “administering” as used herein is interchangeable with “applying” or “treating.”

Unless otherwise specified, the term “hyperbranched” as used herein is interchangeable with “dendritic.”

Unless otherwise specified, the term “treatment group” as used herein refers to groups including “PD10 group,” “PD20 group,” “PD50 group,” and “commercial product group.”

As used herein, the term “polymer dots (PDs)” includes carbon dots with non-conjugated structures.

As used herein, the term “polymer dots (PDs)”includes carbon nanodots (CNDs).

As used herein, the term “nanozyme” refers to nanoparticles exhibiting enzyme-like catalytic properties. Nanozymes have emerged as a new class of enzymatic nanomaterials that offer higher stability and more readily tunable catalytic activity compared to those of natural enzymes.

As used herein, the term “nanodot” or “nanocluster (NC)” refers to ultrasmall nanoparticles with a core size of less than 10 nm. When particle size of the nanoparticle falls below 10 nm, significant quantization occurs in energy band. To distinguish these quantum-sized nanoparticles from conventional nanoparticles, the term “nanocluster” is typically used in the art to refer the ultrasmall nanoparticles.

As used herein, the term “hybrid cluster” refers to nanoclusters formed from carbon materials (e.g., carbon nanodots) and metals (e.g., metal nanoclusters). These hybrid clusters exhibit spectral characteristics distinct from other nanoparticles and possess unique optical properties.

In some embodiments, the use of polymer dots includes topically administering carbon dots to skin of a subject in need thereof.

In some embodiments, the carbon dots may be hyperbranched carbon dots.

In some embodiments, the hyperbranched carbon dots may have a non-conjugated structure.

In some embodiments, the hyperbranched carbon dots may include a first monomer represented by formula I: A₂B₃ (I), and A₂ may be a dianhydride and B₃ may be a polyetheramine.

In some embodiments, the dianhydride may include a bridged bicyclic alkene structure or a non-alkene structure.

In some embodiments, terminal of the hyperbranched carbon dots may include an amino group, and the first monomer may be aliphatic.

In some embodiments, the hyperbranched carbon dots may include a second monomer represented by formula II: AB₂ (II), the A may be an amino group and the B₂ may be a carboxylic acid; or the A may be the carboxylic acid and the B₂ may be the amino group.

In some embodiments, the hyperbranched carbon dots may include a second monomer represented by formula II: AB₂ (II), the A may be the carboxylic acid and the B₂ may be the amino group.

In some embodiments, an interior of the hyperbranched carbon dots may include an alkene structure.

In some embodiments, the hyperbranched carbon dots may include an amino group structure.

In some embodiments, a terminal of the hyperbranched carbon dots may include a carboxylic group structure.

In some embodiments, the second monomer may be aliphatic.

In some embodiments, the composition further may include a metal clusters.

In some embodiments, the composition may include a hybrid cluster.

In some embodiments, the synthetic yield of the hyperbranched polymer dot used herein may range from 5% to 95%, 20% to 90%, 30% to 80%, or 40% to 70%, for example: about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95%, but the present disclosure is not limited thereto.

In some embodiments, the particle size of the hyperbranched polymer dot used herein may range from 1.5 nm to 15 nm, 2 nm to 10 nm, or 2.5 nm to 6.5 nm, for example: about 1.5 nm, about 2.0 nm, about 2.5 nm, about 3.0 nm, about 3.5 nm, about 4.0 nm, about 4.5 nm, about 5.0 nm, about 5.5 nm, about 6.0 nm, about 6.5 nm, about 7.0 nm, about 7.5 nm, about 8.0 nm, about 8.5 nm, about 9.0 nm, about 9.5 nm, about 10.0 nm, about 10.5 nm, about 11.0 nm, about 11.5 nm, about 12.0 nm, about 12.5 nm, about 13.0 nm, about 13.5 nm, about 14.0 nm, about 14.5 nm, or about 15.0 nm, but the present disclosure is not limited thereto.

In some embodiments, the polymer carbon dot used herein acts as nanozymes with biological activity and serves as materials for enhancing hair growth and hair follicle regeneration.

In some embodiments, the hyperbranched polymer dot may be combined with the metal cluster to form the nanozyme of the hybrid cluster that possess antioxidant and/or anti-inflammatory properties.

### EXAMPLES

Exemplary embodiments of the present disclosure are further described in the following examples, which should not be construed to limit the scope of the present disclosure.

### Materials and Methods

Preparation of hyperbranched Polymer Dots

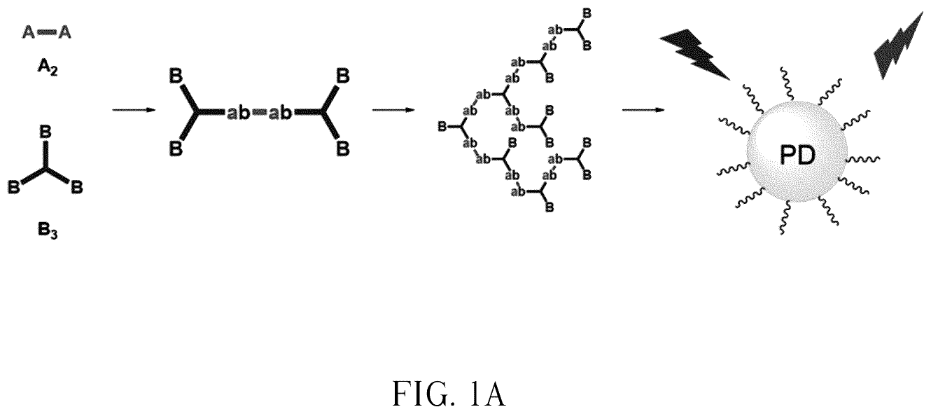

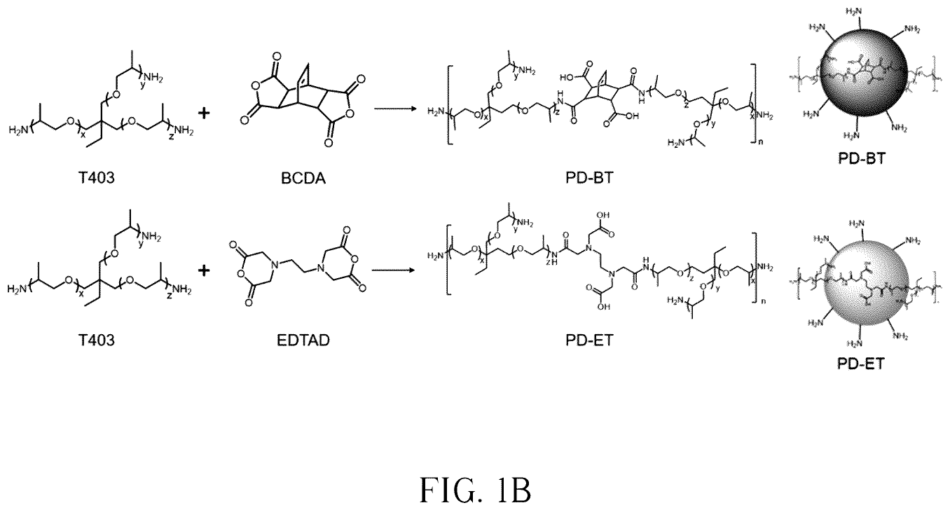

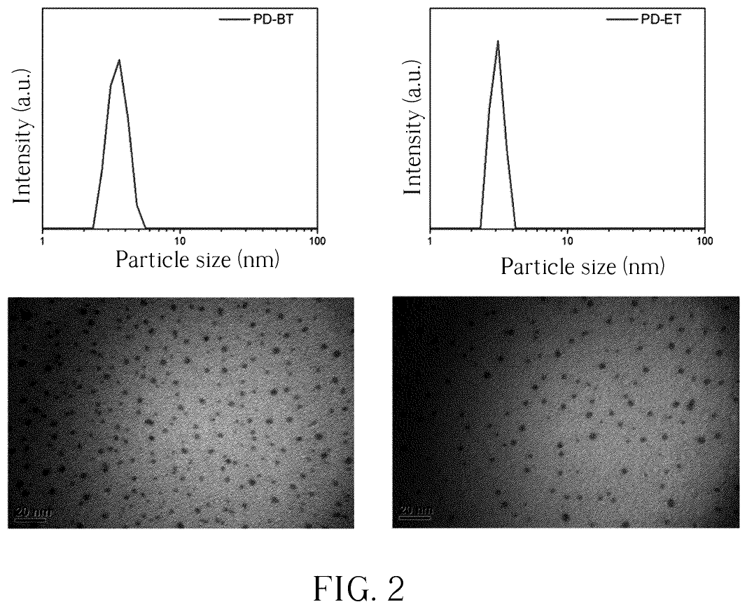

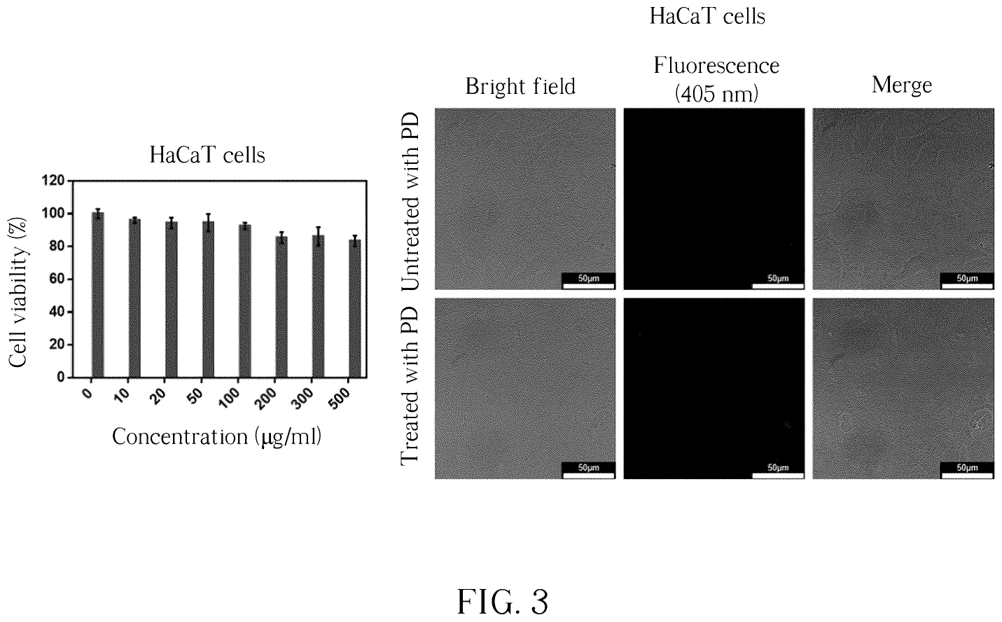

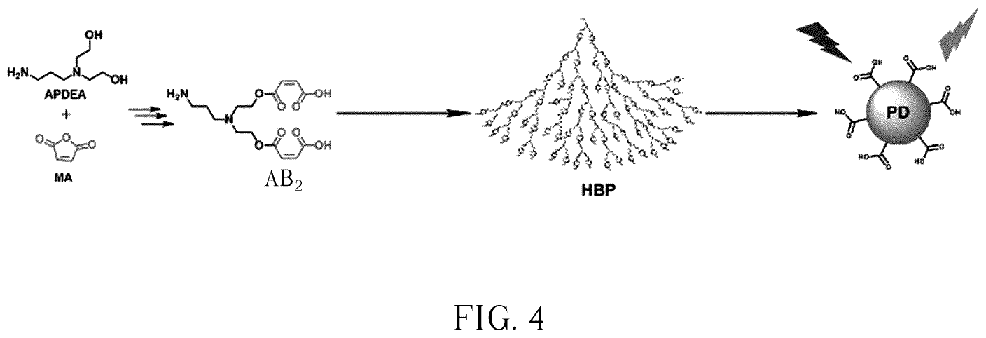

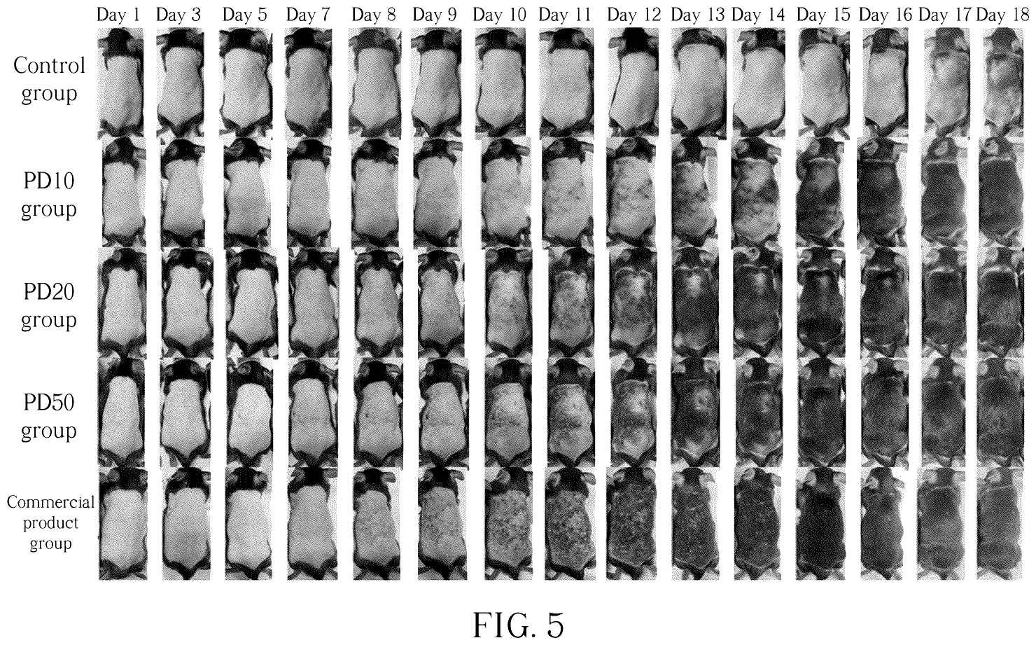

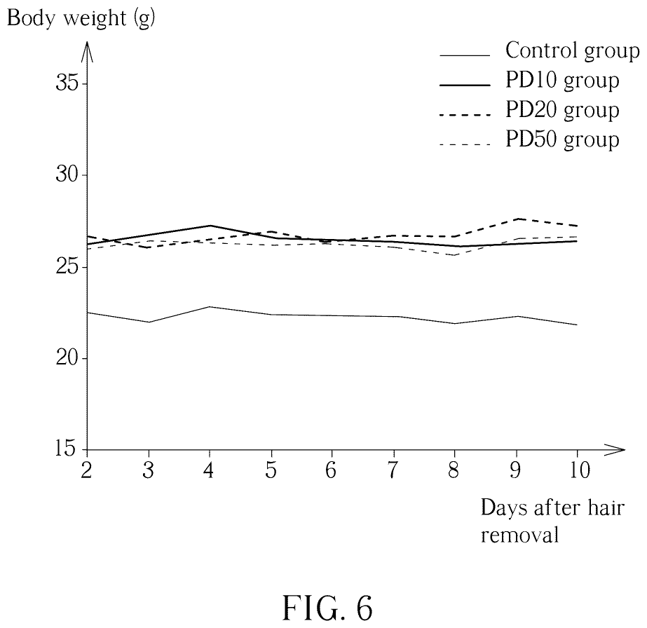

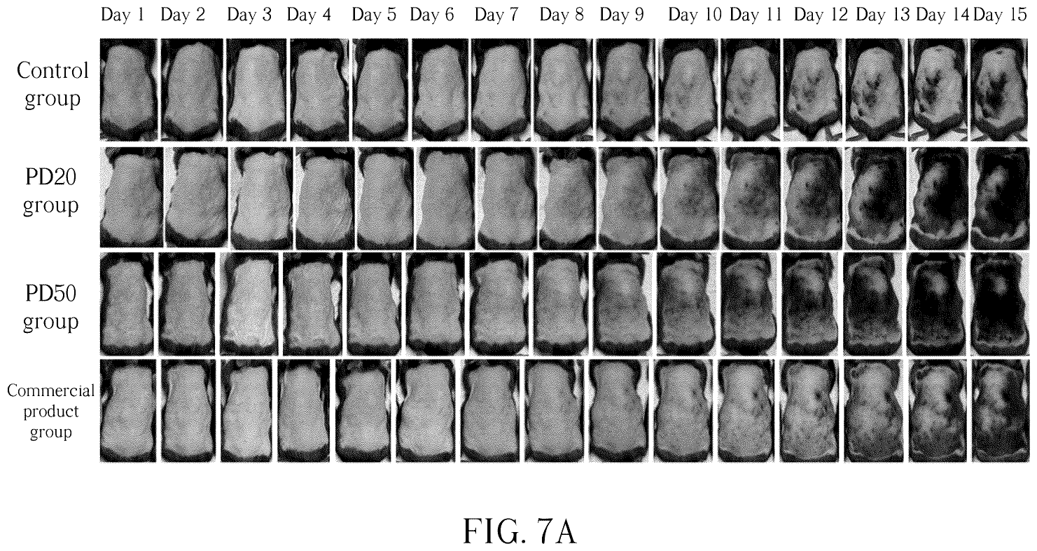

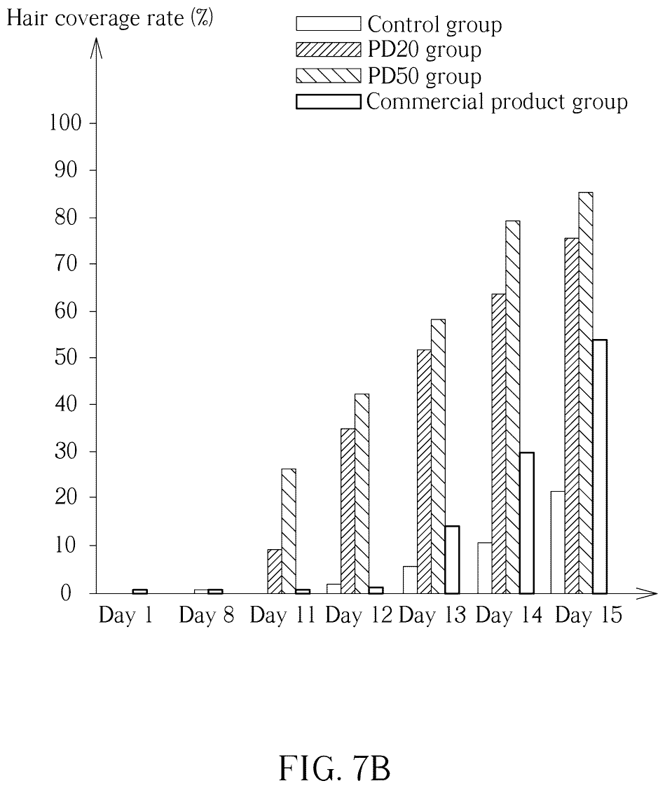

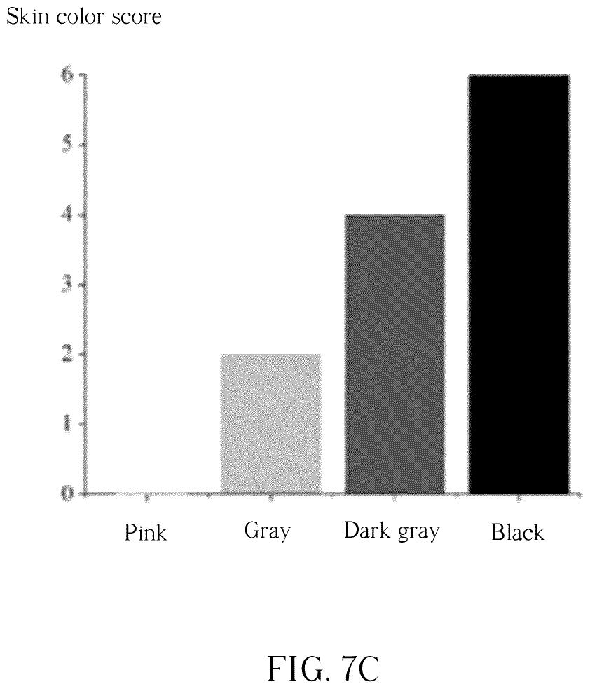

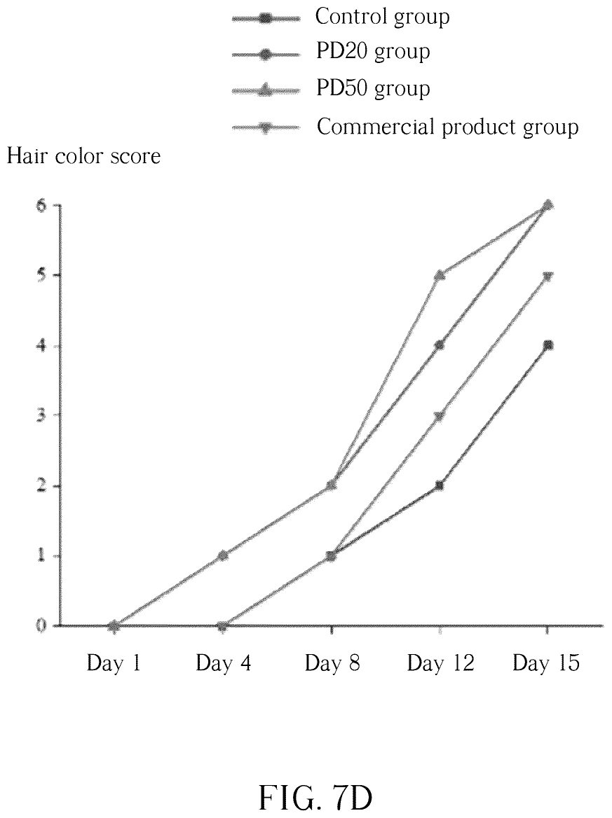

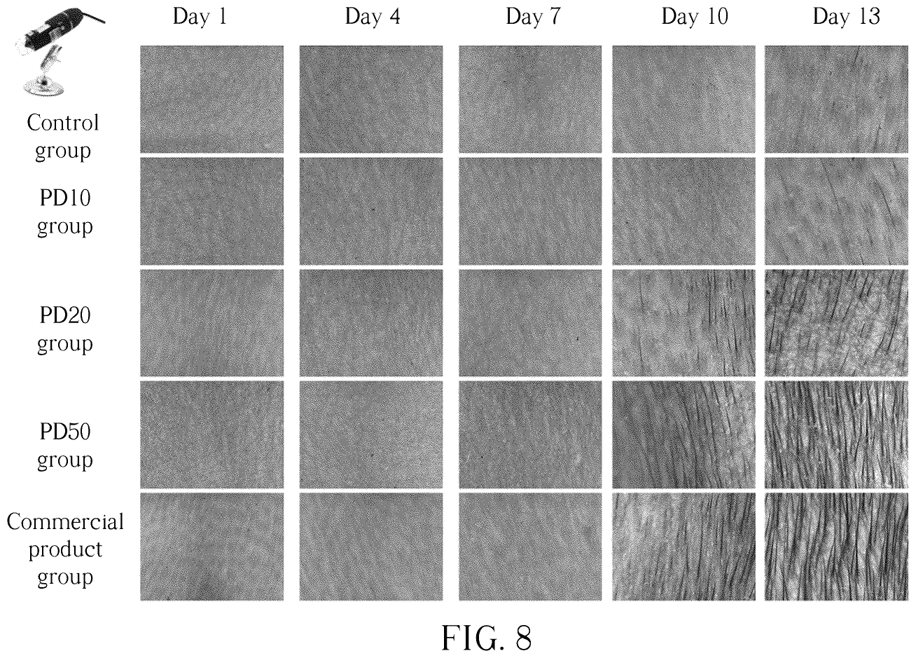

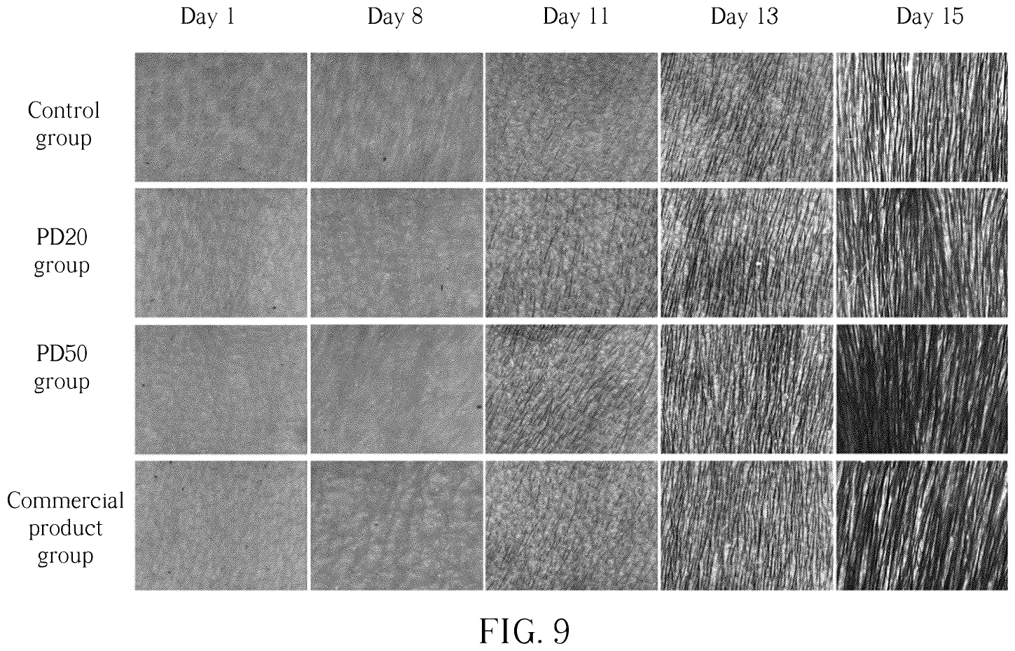

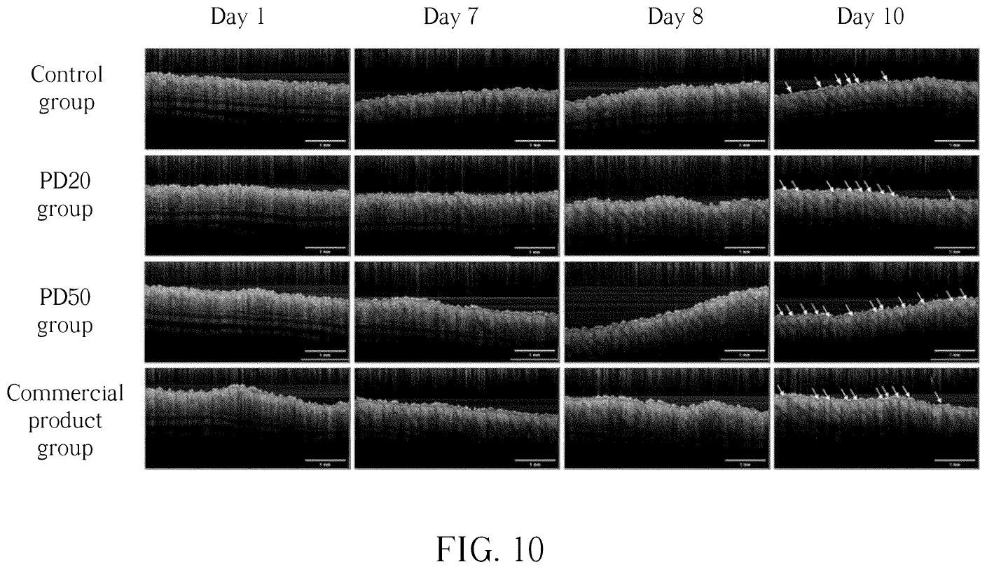

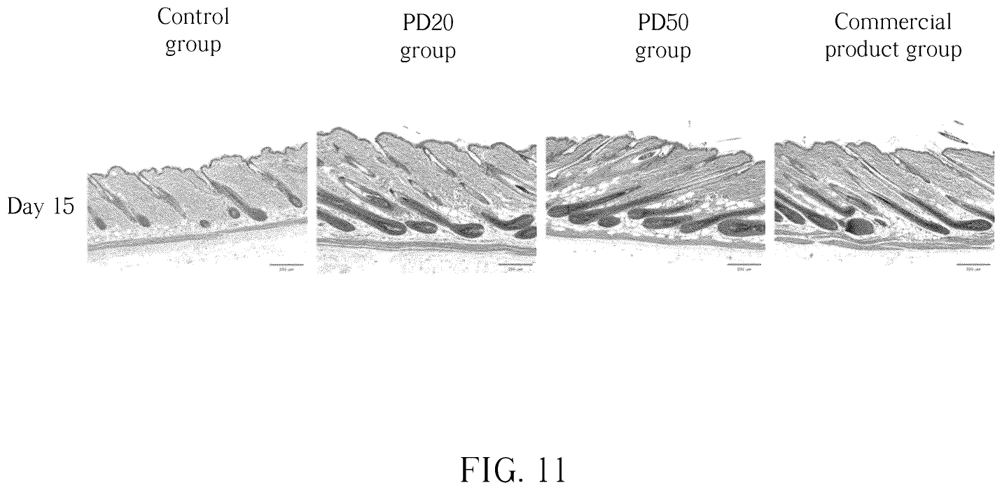

Two types of hyperbranched polymer dots are synthesized using an A₂+B₃ composition structure including wholly aliphatic monomers (FIG. 1A), which are polymers possessing the aliphatic group and alicyclic group with non-conjugated hyperbranched structures. Two types of dianhydrides are used as the A2 component: a bridged bicyclic alkene (e.g., bicyclo[2.2.2]oct-7-ene-2,3,5,6-tetracarboxylic dianhydride (BCDA)) and a non-alkene structure (e.g., ethylenediaminetetraacetic dianhydride (EDTAD)); and polyetheramine (Jeffamine® T403, hereinafter referred to as T403 in the present disclosure and in FIG. 1B) is used as the B₃ component. The aforementioned two types of hyperbranched polymer dots exhibit a non-conjugated hyperbranched poly(amido acid) (also referred to as poly(amino acid)) structure with terminal amino functional group. The hyperbranched polymer dots synthesized using the bridged bicyclic alkene or non-alkene are hereinafter referred to as PD-BT (the upper panels of FIG. 1B) or PD-ET (the lower panels of FIG. 1B), respectively.

In some embodiments, PD-BT and PD-ET exhibit non-conventional fluorescence. The fluorescence intensity of PD-ET is affected by pH value, whereas the rigid aliphatic bridged bicyclic structure of PD-BT allows itself to be less sensitive to pH value changes. The net charges of PD-BT and PD-ET may be adjusted by varying the pH value. Quantum yields (QY) of the PD-BT and the PD-ET are 12.8% and 14.0%, respectively, under 435 nm and 438 nm emission (em) wavelengths.

The particle sizes of PD-BT and PD-ET analyzed by dynamic light scattering (DLS) are about 3 nm to 5 nm (the upper panels of FIG. 2; “a. u.” stands for arbitrary unit); and the morphologies of PD-BT (the lower left panel of FIG. 2) and PD-ET (the lower right panel of FIG. 2) are observed by high-resolution transmission electron microscopy (HRTEM).

In some embodiments, PD-BT at a concentration of 50 μg/mL or PD-ET at a concentration of 500 μg/mL exhibits no cytotoxicity toward human breast cancer cells (MCF-7 cells) and/or human keratinocytes (HaCaT cells).

As shown in the left panel of FIG. 3, HaCaT cell viability is assessed using the MTT assay (3-(4,5-dimethylthiazol-2-yl)-2,5-diphenyltetrazolium bromide). After 48 hours of treatment with various concentrations of PD-ET of the present disclosure, HaCaT cell viability is measured to evaluate the cytotoxicity of the polymer dot of the present disclosure.

As shown in the right panels of FIG. 3, the HaCaT cells treated with PD-ET (20 μg/mL) for 6 hours uptake the PDs of the present disclosure (i.e., the PDs of the present disclosure are located in the cells) analyzed using confocal microscopy, and the PDs of the present disclosure may emit stable fluorescence signal without exhibiting photobleaching effect. The “merge” in the right panels of FIG. 3 represents an overlay of “bright field” image and “fluorescence”image of PDs of the present disclosure excited at 405 nm.

An AB₂ monomer structure is synthesized using N-(3-aminopropyl)diethanolamine (APDEA) and maleic anhydride (MA). The AB2 monomers undergo self-polymerization to form a hyperbranched polymer (HBP), and self-assemble into hyperbranched polymer dots, exhibiting a quantum yield (QY) of 13.5% at an emission (em) wavelength at 460 nm with and a particle size of about 9.1 nm (FIG. 4).

Establishment of an Animal Model with Hair Follicles under Telogen Phase

To establish an animal model with hair follicles under telogen phase (the dorsal regions of the mice are pink), the hair of the dorsal regions of 6-to 8-week-old male mice (C57BL/6) are first trimmed using electric clippers and then removed using a depilatory. Based on PD dosage, the mice are randomly divided into five groups (4 mice per group): control group (phosphate-buffered saline; PBS); PD10 group (10 mg/mL); PD20 group (20 mg/mL); PD50 group (50 mg/mL); and commercial product group (Minoxidil; 50 mg/mL). Each mouse receives a daily topical application of 150 μL of the PBS (control group), PD (PD10 group, PD20 group, or PD50 group), or commercial product group applied to the dorsal region of the skin using a graduated pipet. Body weight of the mice is measured, and the hair follicle growth on the dorsal region of the skin is monitored and recorded every three days using a digital trichoscope. The aforementioned treatment for examining the effect of enhancing or activating the animal model with hair follicles under telogen phase lasts for either 18 or 15 days, and the mice are sacrificed at day 18 or 15 for analyzing the distribution of PD in the skin. Using the intrinsic fluorescence properties of PD to confirm whether PD is uniformly distributed and has reached the targeted skin regions for exerting its effects by fluorescence microscopy.

In at least one embodiment of the present disclosure, hyperbranched polymer dots may enhance or activate hair growth in an animal model with hair follicles under telogen phase. In some embodiments, the animal model may be mice aged 6 to 8 weeks, but the present disclosure is not limited thereto.

In at least one embodiment of the present disclosure, hyperbranched polymer dots may prevent hair loss in an animal model with hair follicles under the telogen phase. In some embodiments, the animal model may be mice aged 6 to 8 weeks, but the present disclosure is not limited thereto.

Establishment of Animal Model with Hair Follicles under Prolonged Telogen Phase

To establish an animal model with hair follicles under prolonged telogen phase, the hair of the dorsal regions of 60-week old aged male mice (C57BL/6) are first trimmed using electric clippers and then removed using a depilatory. In addition, based on PD dosage, the aforementioned hair removed aged mice are randomly divided into five groups (4 mice per group): control group (PBS); PD10 group (10 mg/mL); PD20 group (20 mg/mL); PD50 group (50 mg/mL); and commercial product group (Minoxidil; 50 mg/mL). Each aged mouse receives a daily topical application of 150 μL of the PBS (control group), PD (PD10 group, PD20 group, or PD50 group), or commercial product group applied to the dorsal region of the skin using a graduated pipet. Body weight of the aged mice is measured, and the hair follicle growth on the dorsal region of the skin is monitored and recorded every three days using a digital trichoscope. The aforementioned treatment for examining the effect of enhancing or activating the animal model with hair follicles under telogen phase lasts for 18 days, and the aged mice are sacrificed at day 18 analyzing the distribution of PD in the skin. Using the intrinsic fluorescence properties of PD to confirm whether PD is uniformly distributed and has reached the targeted skin regions for exerting its effects by fluorescence microscopy.

In at least one embodiment of the present disclosure, hyperbranched polymer dots may enhance or activate hair growth in an animal model exhibiting hair follicle degeneration or atrophy associated with a prolonged telogen phase. In some embodiments, the animal model may be 60-week old aged mice, but the present disclosure is not limited thereto.

In at least one embodiment of the present disclosure, hyperbranched polymer dots may prevent hair loss in an animal model exhibiting hair follicle degeneration or atrophy associated with a prolonged telogen phase. In some embodiments, the animal model may be 60-week old aged mice, but the present disclosure is not limited thereto.

### Analysis and Results

As shown in FIG. 5, various concentrations of PD are topically applied daily to the dorsal region of the skin of mice under the telogen phase, and hair growth is monitored for 18 days. Hair growth is progressively enhanced with increasing PD concentration. At day 15 of the treatment, hair growth of the PD20 group, PD50 group, and commercial product group does not show any significant different, with PD50 group exhibiting hair growth effect closely resembling to that of the commercial product group. As shown in FIG. 6, there are no significant changes in body weight across all groups during the 18-day testing period, indicating that administration of different concentrations of the PD does not cause physical discomfort or other side effects in the mice.

As shown in FIG. 7A to FIG. 7D, various concentrations of PD are topically applied daily to the dorsal region of the skin of mice under telogen phase, and hair growth is monitored for 15 days. Following hair removal, the dorsal region of the skin appears to be pink due to the hair follicles under telogen phase. At day 8 of the treatment, the dorsal region of the skin of mice in PD20 group and PD50 group have turned gray, indicating a transition of the hair follicles into the anagen phase. Using the image analysis software ImageJ, hair coverage rates on the dorsal region of the skin are quantified. Rapid hair growth is observed at day 11 in the PD20 group and PD50 group, with hair coverage rates of 9% and 26%, respectively. In contrast, the commercial product group exhibits noticeable hair growth only by day 13, with the hair coverage rate of 14%. Moreover, the control group exhibits only 5% hair coverage rate. At day 15 of treatment, the PD20 group and PD50 group have higher hair coverage rate compared with that of the control group (21%), with enhanced follicular regeneration observed as PD concentration increased. In addition, the PD20 group has a hair coverage rate of 75%, which is significantly higher than that of the commercial product group (53%).

Since the skin color of the hair removal dorsal region of mice changes in response to hair follicle regeneration, transitioning from pink (telogen phase) to gray and eventually to black, skin color may serve as an indicator of level of hair follicle growth. The skin color of the dorsal region of the mice is scored on a scale from 0 to 6, corresponding to pink, gray, dark gray, and black, and the quantified results of skin color scores of the dorsal region of the mice (FIG. 7C and FIG. 7D) are consistent with the hair coverage rates observed (FIG. 7B).

As shown in FIG. 8, a trichoscope is used to observe dorsal hair growth in mice across all groups under hair follicle telogen phase treated with various concentrations of PD for 18 days.

As shown in FIG. 9, a trichoscope is used to observe dorsal skin surface and hair growth over 15 days in mice with hair follicles under telogen phase treated with various concentrations of PD. At day 11, the control group exhibits low and limited hair volume, and the commercial product group show shorter hair compared with the PD20 and PD50 groups. In addition, as shown in FIG. 10, optical coherence tomography (OCT) is used to scan the dorsal skin of live mice, and the imaging depth reaches up to 0.5 mm, enabling clear visualization of the epidermis, upper dermis, and hair follicles, while minimizing noise caused by breathing and heartbeat. At day 1, the OCT images indicate that the hair follicles are under telogen phase. A substantial increase in hair follicle formation is observed in the PD50 group, followed by the PD20 group started at day 8. In contrast, the commercial product group exhibits fewer hair follicles, and the control group shows significantly fewer or almost no new hair follicles. After 10 days of treatment, imaging of the upper dermis is limited due to hair shafts protruding from the skin surface, interfering the scanning depth. As shown in FIG. 11, to confirm the effect of PD on hair follicle regeneration, dorsal skin tissue is sectioned on day 15, followed by hematoxylin and eosin (H&E) staining to evaluate follicular morphology changes. In the control group, hair follicles are primarily located in the dermis and exhibits slower hair follicle growth progression compared with the PD20, PD50, and commercial product groups. In treated groups (PD20 group, PD50 group and commercial product group), hair follicles have extended into the subcutaneous tissue (i.e., indicating accelerated development) and exhibit larger diameters and greater average lengths of the hair follicles compared with those in the control group.

Those skilled in the art will readily observe that numerous modifications and alterations of the embodiments may be made while retaining the teachings of the invention. Accordingly, the above disclosure should be construed as limited only by the metes and bounds of the appended claims.

What is claimed is:

1. A use of a polymer dot in manufacture of a composition for enhancing hair growth or preventing hair loss, wherein the polymer dot comprises a carbon dot.

2. The use of claim 1, further comprising topically administering the carbon dot to skin of a subject in need.

3. The use of claim 1, wherein the carbon dot is a hyperbranched carbon dot.

4. The use of claim 3, wherein the hyperbranched carbon dot is a non-conjugated structure.

5. The use of claim 4, wherein the hyperbranched carbon dot comprises a first monomer represented by Formula (I): A₂B₃ (I), wherein A₂ is a dianhydride, and B₃ is a polyetheramine.

6. The use of claim 5, wherein the dianhydride comprises a bridged bicyclic alkene structure or a non-alkene structure.

7. The use of claim 5, wherein a terminal of the hyperbranched carbon dot comprises an amino group, and the first monomer is aliphatic.

8. The use of claim 4, wherein the hyperbranched carbon dot comprises a second monomer represented by Formula (II): AB₂ (II), wherein
A is an amino group and B₂ is a carboxylic acid.

9. The use of claim 4, wherein the hyperbranched carbon dot comprises a second monomer represented by Formula (II): AB₂ (II), wherein
A is a carboxylic acid and B₂ is an amino group.

10. The use of claim 8, wherein an interior of the hyperbranched carbon dot comprises an alkene structure.

11. The use of claim 8, wherein the hyperbranched carbon dot comprises an amino group structure.

12. The use of claim 8, wherein a terminal of the hyperbranched carbon dot comprises a carboxyl group structure.

13. The use of claim 8, wherein the second monomer is aliphatic.

14. The use of claim 9, wherein an interior of the hyperbranched carbon dot comprises an alkene structure.

15. The use of claim 9, wherein the hyperbranched carbon dot comprises an amino group structure.

16. The use of claim 9, wherein a terminal of the hyperbranched carbon dot comprises a carboxyl group structure.

17. The use of claim 9, wherein the second monomer is aliphatic.

18. The use of claim 1, wherein the composition further comprises a metal cluster.
